(19) United States
(12) Patent Application Publication
(10) Pub. No.: US 2008/0262527 A1
(43) Pub. Date: Oct. 23, 2008

(54) METHOD AND APPARATUS FOR ASSISTING IN THE INTRODUCTION OF SURGICAL IMPLEMENTS INTO A BODY

(76) Inventors: Joseph Charles Eder, Los Altos Hills, CA (US); Peter Seth Edelstein, Menlo Park, CA (US)

Correspondence Address: GLENN PATENT GROUP, 3475 EDISON WAY, SUITE L, MENLO PARK, CA, 94025, US

(21) Appl. No.: 12/165,389

(22) Filed: Jun. 30, 2008

Related U.S. Application Data
(63) Continuation-in-part of application No. 11/848,015, filed on Aug. 30, 2007.
Application No. 11/848,015 is a continuation-in-part of application No. 11/781,159, filed on Jul. 20, 2007.
Application No. 11/781,159 is a continuation-in-part of application No. 29/304,348, filed on Feb. 28, 2008.
(60) Provisional application No. 60/824,393, filed on Sep. 1, 2006.

(51) Int. Cl. A61B 17/34

(52) U.S. Cl. 606/185

(57) ABSTRACT

A tissue penetration device is provided that includes a bell-like, at least partially transparent housing. A valved port is provided in the housing for introduction of a penetrator therein. The valved port includes both a valve control and a port. A vacuum system, including a vacuum source, is securely and sealably attached through the housing to advance a patient's tissue onto the penetrator.

### CROSS-REFERENCE TO RELATED APPLICATION

The application is a continuation-in-part of U.S. patent application Ser. No. 11/848,015, filed 30 Aug. 2007, is a continuation-in-part of patent application Ser. No. 11/781,159, filed 20 Jul. 2007, which claims priority from U.S. [provisional patent application Ser. No. 60/824,393, filed Sep. 1, 2006, and is a continuation-in-part of U.S. design patent application Ser. No. 29/304,348, filed 28 Feb. 2008, all of which are incorporated herein in their entirety by this reference thereto.

### BACKGROUND OF THE INVENTION

1. Technical Field

The invention relates to surgical implements. More particularly, the invention relates to a method and apparatus for assisting in the introduction of surgical implements into a body.

2. Description of the Prior Art

Significant morbidity and mortality occurs each year by iatrogenic injuries during establishment of a pneumoperitoneum prior to the performance of laparoscopic surgical procedures. The main source of these injuries is inadvertent perforation of blood vessels, intestines, or other viscera within the abdominal (peritoneal) cavity when the penetration device (needle, trocar, biopsy instrument, etc.) is advanced too far through the abdominal wall, inadvertently piercing the underlying organs that are located adjacent thereto. These injuries are more common with inexperienced surgeons, but can occur even in the most experienced hands. Built-in safety devices exist in the perforation devices themselves, but injuries still occur because of the close proximity of the structures that are intended to be perforated and those to be avoided. In addition, significant time, resulting in additional anesthetic time (and consequences) for surgical patients, results from failure to pass the penetration device (needle, trocar, biopsy instrument, etc.) far enough. In such situations, the device tip is incorrectly positioned within the abdominal wall, superficial to the target abdominal (peritoneal) cavity. This failure to position the device correctly requires time to recognize and additional time to correct and verify. During these corrective maneuvers, the patient receives anesthetic.

Vacuum has been used to fix or distort the body and body cavities. In addition, techniques exist for insufflation or mechanical or vacuum elevation of the abdominal wall during surgical procedures.

In U.S. Pat. No. 6,042,539, a vacuum-actuated tissue-lifting device and method for performing a surgical procedure in an operative space of a patient are disclosed. The preferred device has a shell with a profile configured to surround a tissue surface of the patient, a vacuum port located on the shell for applying a vacuum between the shell and the tissue surface, and an air conduit extending through the shell to permit air to pass into the operative space of the patient when vacuum is applied.

In U.S. Pat. No. 6,340,358, a trocar is disclosed having a safety shield control mechanism that prevents the inner cannula from rotating and from moving axially when in the locked position. The safety shield control mechanism applies consistent pressure on the safety shield and has an open architecture for ease of sterilization. The trocar provides holding levels for different sizes of hands.

In U.S. Pat. No. 6,197,041, a pneumatically powered trocar assembly is disclosed that includes a source of compressed gas which releases a metered amount of gas to a chamber. A piston slidably positioned within the chamber is driven forward by the compressed gas introduced therein, and an obturator with a tissue piercing tip are advanced thereby. Optionally, a sensor detects the presence of body tissue within the cutting path of the tip and blocks the passage of compressed gas to the chamber, or alternatively, opens an escape vent to release compressed gas therefrom if insufficient body tissue resistance is encountered.

In U.S. Pat. No. 5,669,883, a Veress needle and cannula assembly is disclosed that includes a stainless steel cannula assembly with a cannula having an outer diameter of approximately 4 mm and a Veress needle assembly having a Veress needle with an outer diameter of approximately 3 mm. The cannula assembly includes a proximal valve assembly and the Veress needle is insertable through the valve assembly.

In U.S. Pat. No. 5,690,607, an apparatus is disclosed for allowing two retractors to be used to lift the abdominal wall to provide improved visualization and working space in the abdomen of obese patients, and in the lateral regions of the abdomen of normal patients. The apparatus connects a first retractor and a second retractor to a mechanical lifting arm, and comprises a bar, and first, second, and third connecting devices. The apparatus is used by making a first incision and a second incision in the abdominal wall at separated locations. The first retractor is inserted into the first incision, and the second retractor is inserted into the second incision. The first retractor and the second retractor are attached to the crossbar, anal a lifting force is applied to the crossbar.

In U.S. Pat. No. 5,575,759, an apparatus for retracting an organ to gain access to treat a tissue is disclosed. The apparatus has a main envelope, a second envelope, a first inflation device and a second inflation device. The main envelope encloses a main chamber, and includes a window and a removable window. The second envelope covers substantially all the main envelope, except the window and the removable window. The second envelope and the main envelope enclose a second chamber outside the main chamber. The first inflation device passes a fluid into the main chamber to expand the main chamber and the second chamber from a compacted state to retract the organ. The second inflation device passes a fluid into the second chamber to further expand the second chamber to maintain the organ in its retracted state after fluid has been released from the main chamber.

In U.S. Pat. No. 5,562,603, an apparatus is described for laparoscopically retracting an organ inside the body to provide surgical access to adjacent tissue. The apparatus includes a thin, flexible envelope, which encloses a chamber. The envelope is laparoscopically insertable in a collapsed state into a body cavity, and the chamber is inflatable to an expanded state following introduction of the envelope into the body. Inflation of the chamber causes retraction of adjacent tissue. An elastomeric seal is insertable into the chamber following inflation and is attachable to part of the envelope inside the chamber following inflation of the chamber. The seal provides a gas-tight seal to maintain the chamber in the expanded state, and to maintain the organ in the retracted state, notwithstanding the piercing of an aperture in the part of the envelope covered by the seal.

In U.S. Pat. No. 5,531,856, an inflatable apparatus for organ retraction includes a main envelope that forms a main chamber is disclosed. An additional chamber is formed by attaching the periphery of an additional envelope to the outside or the inside of the main envelope. The part of the surface of the main envelope that is not covered by the additional envelope provides a plurality of windows, which, after the additional chamber is inflated, may be at least partially removed to provide apertures through, which treatment or observation can be carried out.

In U.S. Pat. No. 5,527,264, a method is disclosed for retracting an organ inside the body to provide access for treating a tissue, a retractor having a main envelope, which defines a main chamber is positioned in a collapsed state adjacent to the organ to be retracted. The main chamber is subsequently inflated to retract the adjacent organ. A surgical instrument is passed through the main envelope into the main chamber to contact the tissue for treatment. In U.S. Pat. No. 5,522,790, a first inflatable retraction device is disclosed having a first inflatable chamber and a non-pressurized chamber inside the main chamber. The non-pressurized chamber is expanded by inflating a second inflatable chamber. The non-pressurized chamber enables the main chamber to remain inflated when an aperture is cut in the envelope of the main chamber, through which treatment is carried out. A second inflatable retraction device has an inflatable retractor and a maintainer. The inflatable retractor retracts the organ and the maintainer maintains the organ in its retracted condition after the inflatable retractor is deflated. The maintainer can be inflatable, and can be inside or outside the inflatable retractor. A self-retracting endoscope has an optical assembly with an expandable retractor fitted to its distal end. The distal end of the endoscope is inserted into the body with the retractor in a collapsed condition. The retractor is then expanded to retract organs that would otherwise obstruct the view from the distal end of the optical assembly. After observations are complete, the retractor is returned to its collapsed condition. An insertion tube enables cylindrical objects, such as packaged inflatable retraction devices, to be pulled, instead of pushed, into the body. The additional chamber of an inflatable retraction device having two inflatable chambers is filled with a slurry of a particulate solid in a liquid. The liquid is removed and the additional chamber evacuated to consolidate the particulate solid. This increases the retracting strength of the additional chamber.

In U.S. Pat. No. 5,505,689, a fan retractor is disclosed for laparoscopic surgery has a pair of angle-shaped elements with first legs disposed in parallel relationship to one another and second legs extending laterally from the first legs for movement between a juxtaposed collapsed condition and a fanned-out expanded condition responsive to rotation of the first legs about their longitudinal axes. Actuators are provided on the first legs to move the second legs between the collapsed and extended conditions. A first lock engages the actuators to lock the second legs in the extended condition and against movement toward or away from one another. A second lock in the form of a block slidably received on the first legs is selectively engageable between the second legs when in the extended condition. When engaged, the second lock serves both to block the second legs from movement toward one another and to restrain the first legs against movement away from one another.

In U.S. Pat. No. 5,465,711, an organ or tissue plane to be retracted is performed to gain access for a surgical instrument to treat an organ or tissue plane to be treated. An inflatable retractor, including a main envelope enclosing a main chamber, is provided with the main envelope in a collapsed state. The main envelope of the retractor is placed adjacent the organ or tissue plane to be retracted. The main chamber is inflated to an expanded state to retract the organ or tissue plane to be retracted. An aperture is pierced in the main envelope to provide access for the surgical instrument passed into the main chamber to contact an organ or tissue plane to be treated while the main chamber is maintained in the expanded state, notwithstanding the aperture pierced in the main envelope.

In U.S. Pat. No. 5,454,367, an inflatable retractor including a main envelope enclosing a main chamber is provided. The main envelope is provided in a collapsed state. An elastomeric window is also provided. The main envelope of the inflatable retractor is placed adjacent the organ inside the body, and the main chamber is expanded to an expanded state to retract the organ. Following inflation of the main chamber to the expanded state, the elastomeric window is attached to the main envelope inside the main chamber to cover part of the main envelope. The surgical instrument is passed into the main chamber. An aperture is pierced in the pan of the main envelope covered by the elastomeric window to provide access for the surgical instrument to contact the tissue. The elastomeric window provides a gas-tight seal to maintain the main chamber in the expanded state.

In U.S. patent application publication no. 20040049127, a system is provided that advances tissue to be perforated onto a stationary perforation device and away from the underlying structures. The simplicity of technique leads to a short learning curve and virtually eliminates the possibility of iatrogenic injuries. In one embodiment, tissue to be perforated is advanced onto a perforation device or piercing instrument. A primary intended use of the such embodiment is taught to be for pulling the abdominal wall onto a perforation device, such as a Veress needle, trocar, or punch biopsy cutting tool, and away from abdominal viscera and great vessels for the initial establishment of a pneumoperiotoneum. Another intended application for such embodiment is for implanting a device below the skin surface for diagnostic or therapeutic purposes.

Referring to FIG. 1 and FIG. 2, which are a side and bottom view, respectively, of a perforation device 10 in accordance with United States patent application publication no. 20040049127, positioned over a non-extended abdominal wall 18. The device 10 comprises a housing 12 having a housing pass-through 12C and a housing seal 12A along a perimeter functioning to form a tight seal between the housing 12 and an abdominal wall 18. The housing 12 is manufactured from a strong non-collapsible material to withstand the internal negative pressure in the range of about 50 to 250 mm Hg exerted therein. Suitable materials are specifically designed for medical use and capable of sterilization. The materials include plastic, plastic composite, rubber, rubber composite, fiberglass, epoxy, glass, glass composite, and the like. Plastic, such as polycarbonate and acrylic materials, and plastic composites are particularly well suited due to their superior strength, transparency, rapid manufacturing and low cost. The housing 12 is usually translucent or transparent. Transparency is preferable to allow the physician to monitor the perforation. Housing 12 is sized to accommodate adults and children of different sizes and/or body mass indexes. The diameter of the circumference of housing 12 in contact with the abdomen for adults ranges from about 3 inches to 8 inches and for pediatric patients from 1½ to 3 inches.

A penetrator 14 is securely and sealably positioned through a top center housing 20 of the housing 12. The seal and fixation of the penetrator 14 can be done by means of the 0-ring 22. The seal and fixation of the penetrator 14 can also be accomplished by other means, e.g. by pressing the operator's fingers on penetrator 14 and braced against the housing pass-through 12C. The penetrator 14 comprises a penetrator device 14C, such as a Veress needle, trocar, or other suitable device, designed to penetrate or cut tissue. The penetrator 14 has a valve 14B for allowing the penetrator tube 14D either to introduce ambient room air, or for connection to a pressurized source of a gas, e.g. carbon dioxide, helium, nitrogen, air, and mixtures thereof, for insufflation. The tissue perforation device 10 further comprises a standard operating room vacuum system 16. A vacuum tube 16A is securely and sealably attached to the housing 12 through a valve or pressure regulator 16B, which functions to regulate the amount of negative pressure exerted within the housing 12.

Referring now to FIG. 3, an extended abdominal wall 18 is shown with an abdominal wall bubble 18A. The abdominal wall bubble 18A is formed by applying negative pressure at vacuum tube 16A, which extends into and is sealed against housing 12. Internal negative pressure within the housing 12 creates an abdominal wall bubble 18A within the housing 10 and elevates the abdominal wall 18 away from the underlying organs and vascular structures. As the abdominal wall bubble 18A enlarges, the abdominal wall 18 is advanced onto the stationary penetrator device 14C. Room air or inert gas introduced through penetrator valve 14B into the peritoneal cavity to further facilitates the separation between the abdominal wall 18 and the underlying organs and vessels. The penetrator device 14C contains an optical device or integral camera 24 at its tip to direct visualization of the passage of penetrator device 14C through tissue. The distance of tissue movement is controlled directly by the operator through regulation of the vacuum. This may be controlled by periodic opening of a vacuum valve, or through a regulator 16B. Optimal vacuum is a function of the tissue characteristics, and is regulated by the operator to achieve the desired tissue displacement. Following penetration and insufflation, the applied vacuum may be released and the housing removed by means of a clamshell housing or by means of an adaptor plate or collar while leaving the perforation device in place through the abdominal wall.

### SUMMARY OF THE INVENTION

A tissue penetration device is provided that includes a bell-like, at least partially transparent housing. A valved port is provided in the housing for introduction of a penetrator therein. The valved port includes both a valve control and a port. A vacuum system, including a vacuum source, is securely and sealably attached through the housing to advance a patient's tissue onto the penetrator.

### BRIEF DESCRIPTION OF THE DRAWINGS

FIG. 1 is a side view of a tissue perforation device positioned over a non-extended abdominal wall;

FIG. 2 is a bottom view of the perforation device of FIG. 1;

FIG. 3 is a side view of the perforation device of FIG. 1 exhibiting an extended abdominal wall forming an abdominal wall bubble;

FIGS. 4A and 4B provide a perspective view (FIG. 4A) and a side elevation view (FIG. 4B) of a vacuum housing incorporating a valved port for introduction of a penetrator according to an embodiment of the invention;

FIGS. 5A and 5B provide a perspective view (FIG. 5A) and a side elevation view (FIG. 5B) of a vacuum housing incorporating a valved port for introduction of a penetrator according to another embodiment of the invention;

FIGS. 6A and 6B provide a side elevation view (FIG. 6A) and a top plan view (FIG. 6B) of a split shell vacuum housing according to a further embodiment of the invention;

FIG. 7 provides a side elevation view of a seal according to a further embodiment of the invention;

FIG. 8 provides a perspective view of a split shell vacuum housing according to a further embodiment of the invention;

FIGS. 9A, 9B, and 9C provide a right side view (FIG. 9A) of a first half of the housing of FIG. 8, a left side view (FIG. 9B) of the half of the housing shown in FIG. 9A, and a top plan view (FIG. 9C) of the housing shown in FIG. 9A;

FIGS. 10A, 10B, and 10C show a right side view of a section of a housing (FIG. 10A), a left side view of the section of housing shown in FIG. 10A (FIG. 10B), and a top plan view (FIG. 10C) of a section of housing shown in FIG. 10A;

FIGS. 11A, 11B, and 11C show a port ceiling mechanism in side elevation view (FIG. 11A), a base seal in perspective view (FIG. 11B) and the port ceiling mechanism of FIG. 11A in top perspective view (FIG. 11C);

FIGS. 12A and 12B show a port seal and side elevation view (FIG. 12A) and a top perspective view (FIG. 12B); and

FIGS. 13A and 13B provide top plan views showing a hinge arrangement for a hinged dome assembly with a 40 degree range of motion (FIG. 13A) and 60 degree range of motion (FIG. 13B).

### DETAILED DESCRIPTION OF THE INVENTION

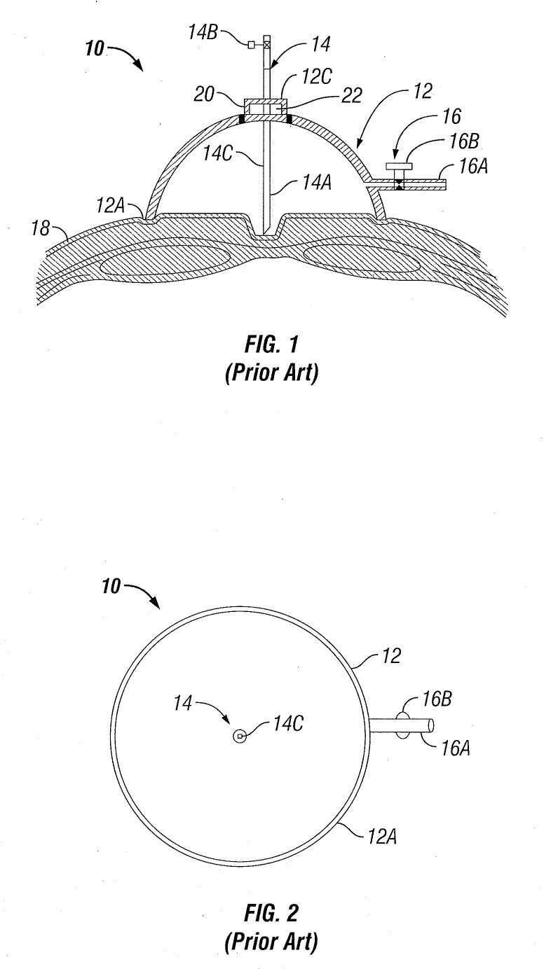

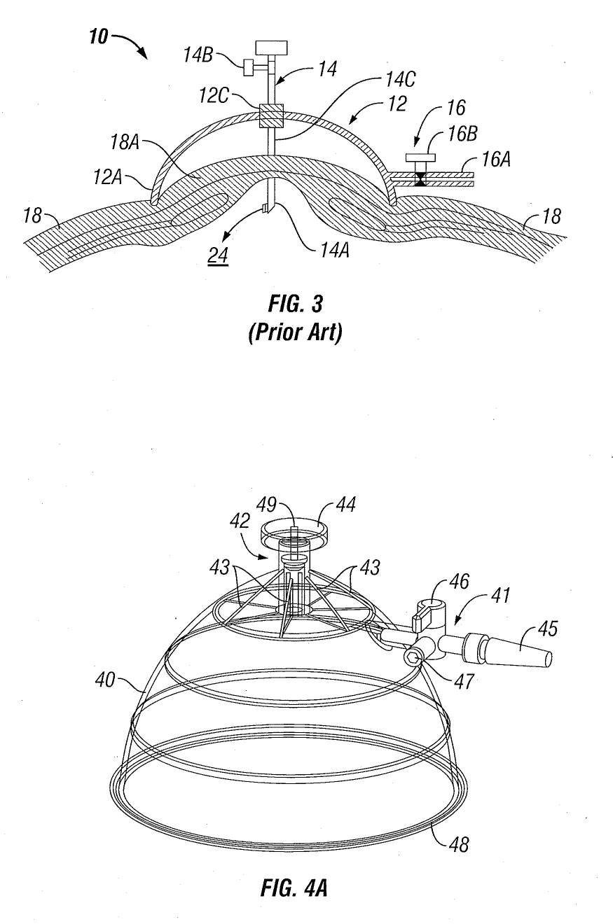

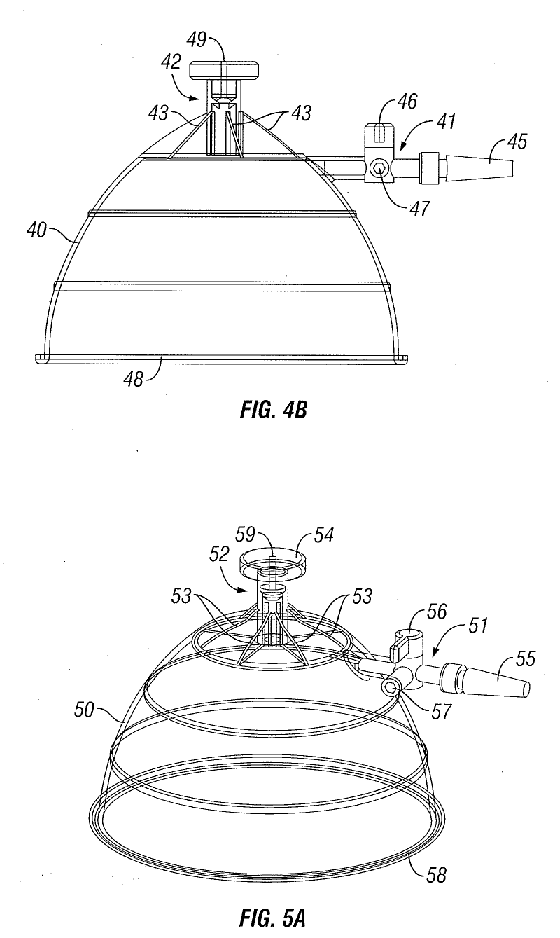

The invention provides several improvements to the prior art device discussed above in connection with FIGS. 1-3.

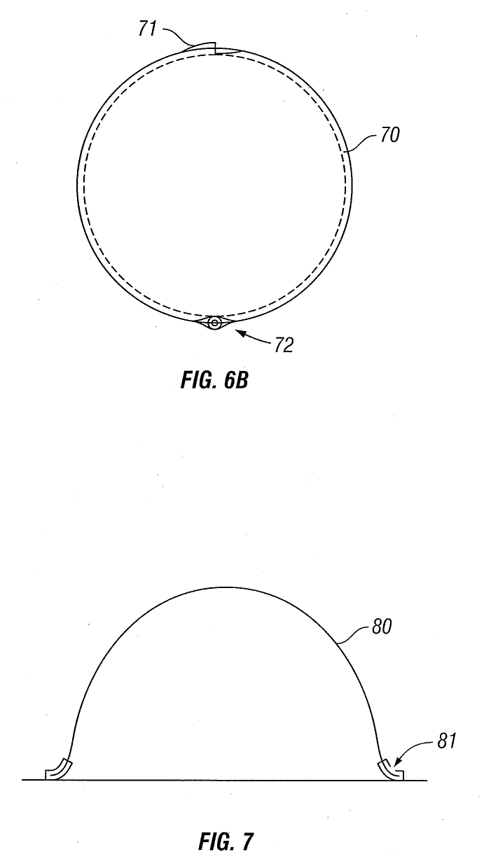

FIGS. 4-A and 4-B provide a perspective view (FIG. 4A) and a side elevation view (FIG. 4B) of a vacuum housing 40 incorporating a valved port 42 for introduction of a penetrator. In FIGS. 4A and 4B, the housing includes a valved port having a valve control 44 and a conduit 49 which allows admission of a needle or trocar or other penetrating device in the housing. The valved port 42 is secured to the housing 40 by a plurality of ribbed extensions 43. The housing 40 also includes a sealing surface 48 for sealing the housing to an individual's skin surface. Additionally, a side-mounted vacuum port 41 is coupled to the housing. The vacuum port 41 incorporates a nipple 45 for attachment to an external source of vacuum and a bleed conduit 47 for allowing entry of air into the housing. A valve control 46 is operable to couple the vacuum to the housing via the vacuum port nipple 45, to purge the vacuum from the housing via the purge port 47, or to seal the vacuum port so that neither the vacuum port nipple 45 nor the purge port 47 are coupled to the interior of the housing 40.

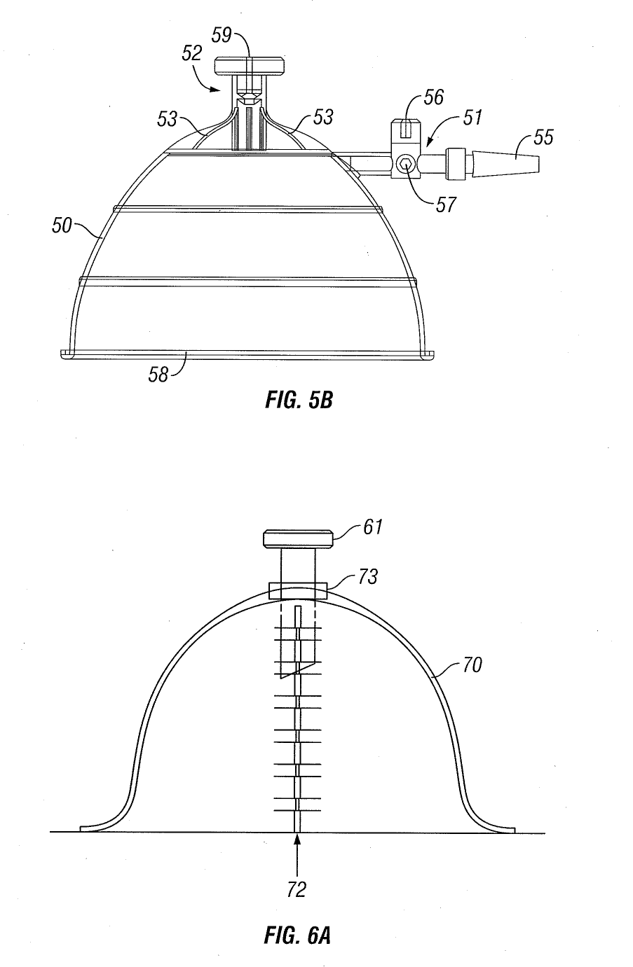

FIGS. 5A and 5B provide a perspective view (FIG. 5A) and a side elevation (FIG. 5B) of a vacuum housing 50 incorporating a valved port 52 for introduction of a penetrator according to another embodiment of the invention. In FIGS. 5A and 5B, a housing 50 includes a valved port 52 which includes a conduit 59 which allows admission of a needle or trocar or other penetrating device into the housing and a valve control 54. In this embodiment of the invention, the ribbed members 53 which support the valved port 52 have a reduced profile which allows a user to grasp and operate the valve control 54 with less effort. As with the embodiment of FIGS. 4A and 4B, the housing 50 includes a sealing surface 58 for sealing the housing to an individual's skin surface. Also included is a vacuum port 51 which incorporates a vacuum port nipple 55, a bleed port 57, and a control valve 56, which operate in the same manner as similar elements discussed above in connection with FIGS. 4A/4B.

Significant in the embodiment of FIGS. 4A/4B and 5A/5B is the provision of a valved port. Accordingly, these embodiments to the invention provide the vacuum port nipple on the side of the housing. This permits ready user access to the valve port control (42; FIGS. 4A/4B and 52; FIGS. 5A/5B). The valve assembly of the valved port is preferably a two or three-way valve. In the open position, the valve allows a vacuum to be created within the housing and in the venting position, the housing is vented to the atmosphere. Thus, when the device is used, for example, to lift a patient's abdominal tissue the valve is set to the closed or vacuum position, and when the device is to be released from the individual, the valve is turned to the open position to release the vacuum.

Significant to the invention is the use of a compression seal fitting for the valved port. One use of the invention is in, for example, laparoscopic procedures and intervascular procedures. A typical top fitting of a compression seal fitting could be used in this embodiment of the invention. Such assembly is a fitting that allows passage of a catheter or guide wire through a port into an artery without loss of vacuum through the port. The top fitting comprises a valve that allows access of a tool through the port, while maintaining a seal having sufficient integrity to prevent the loss of vacuum through the port to the opening through which the tool is introduced. In the invention herein, any fitting is used that allows the introduction of tools through the port once the housing has drawn a vacuum and lifted the patient's abdominal, for example, tissue upwardly into the housing, where the valve may be tightened sufficiently to hold the tool in place, for example to maintain a stationary location for a penetrator while vacuum is applied, such that the tissue is lifted into the penetrator, in one embodiment, by application of vacuum to the housing. The size of tool accommodated by the port is a function of, inter alia, the diameter of the O-ring or other sealing surface within the port of the valve. Thus, in some applications an O-ring may be chosen that is sufficient to seal the port entirely in the absence of a tool therein. In other embodiments, the size of the port is sufficient, when the valve is closed, to prevent most leakage into the housing of the atmosphere as vacuum is applied, and yet provide a sufficient opening to allow introduction of a tool without the risk of damaging the seal and the valve during introduction of the tool therethrough. Thus, the vacuum port may allow for complete sealing of the port, or only partial sealing of the port as desired. In both cases, the valve is adjustable to allow the port to be open sufficiently, in the first case to admit atmosphere into the housing, and in the second case to permit entry therein of a tool.

Because the vacuum port has an adjustment valve, the embodiments of FIGS. 4A/4B and 5A/5B include support ribs, as discussed above. The support ribs are struts to impart sufficient strength to the housing at the point at which the vacuum port is added such that the housing does not break or crack as the valve adjustment is twisted to open and close the port. In some embodiments of the invention, the cylinder portion of the valve is integrally molded with the support in the housing itself to allow manufacture of the housing, support, and valve cylinder as a single assembly. In other embodiments, the vacuum port may be attached to the housing with a washer and nut, or by gluing thereto. In yet other embodiments of the invention, the entire dome of the housing, including the vacuum port could be detachable from the housing, such that different sized ports with different valve arrangements could be interchanged with a common housing.

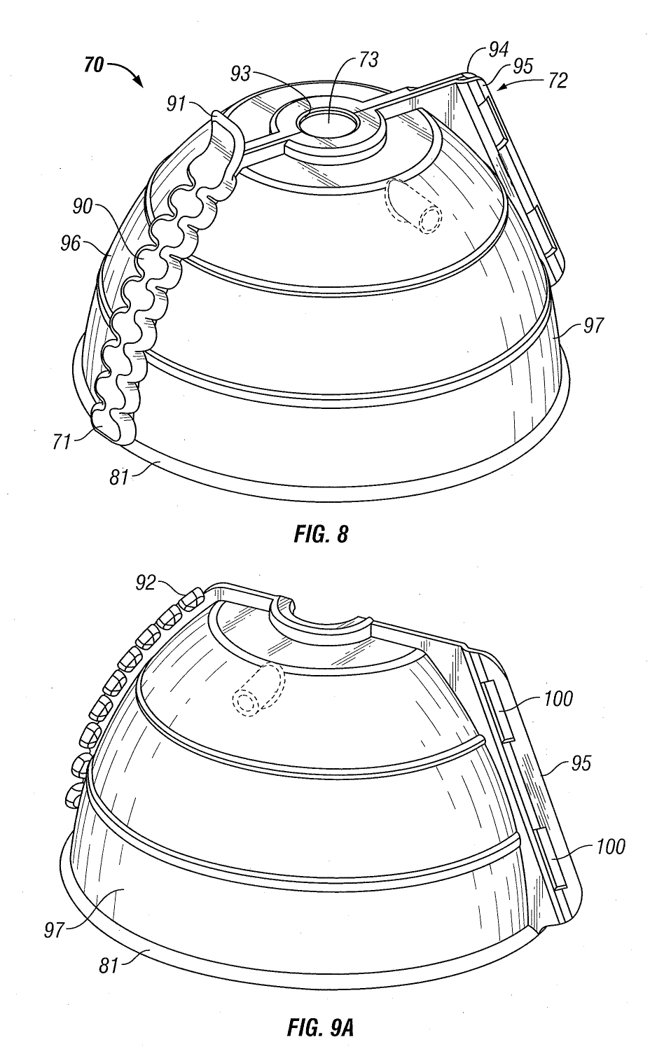

The embodiments of the invention discussed above in connection with FIGS. 4A/4B and 5A/5B may be used with various types of devices such as Veress needles and trocars. In the case of using a Veress needle at a first portion of the procedure, the Veress needle may be used in connection with the housing and vacuum to create a pneumoperitoneum. After the pneumoperitoneum is created, the Veress needle may be removed from the individual and the housing may be lifted away. At that point, a trocar may be inserted into the individual and a laparoscopic or endoscopic procedure may then be pursued. In another embodiment, a trocar may be inserted without first creating a pneumoperitoneum. Once the tissue is drawn upwardly in the dome by application of a vacuum and the body cavity wall is pulled upwardly, the trocar may be introduced into the body tissue or the body tissue may be pulled directly over the trocar. At this point, it is not necessary to remove the trocar until the end of the procedure. In such case, it would be advantageous to remove the housing (see FIGS. 6A/6B discussed below).

As discussed above in connection with FIGS. 4A/4B and 5A/5B, one embodiment includes support ribs that go further up the shaft of the vacuum port (FIGS. 4A/4B) than that of the other embodiment (FIGS. 5A/5B). In either embodiment, the housing maintains the same volume of tissue. However, as discussed above one embodiment provides larger ribs or struts for support, e.g. in a preferred embodiment of about 22 millimeters, as opposed to an embodiment which provides shallower ribs or struts, e.g. in a preferred embodiment of about 15 millimeters. As discussed above, this latter arrangement provides more space for grasping the valve.

FIGS. 6A and 6B provide a side elevation view (FIG. 6A) and a top plan view (FIG. 6B) of a split shell vacuum housing 70 according to a further embodiment of the invention. In this embodiment of the invention, the housing 70 includes a hinge 72 and seal 71, which may be a clasp like seal. The housing has sufficient integrity along the hinge and clasp-like seal to allow maintenance of the vacuum during the initial portion of the procedure when the tissues and skin are lifted away from the internal organs of the patient. As discussed above, a trocar 61 is introduced into the housing through a port 73. Once the trocar has penetrated the tissue of the patient and a pneumoperitoneum (if desired) is established, the housing is opened along the sides, and the halves of the housing are spread apart along the hinge. The housing is then readily removed and the procedure may go forward without the housing being in the way of the surgeon performing the procedure. While a hinge and clasp seal are shown in FIGS. 6A/6B, those skilled in the art will appreciate that other arrangements may be used to provide a housing that may separate into two or more sections. For example, the housing may include a bead and mating groove that allows the sections of the housing to seal together, or the housing may be secured by engaging a groove of a resilient silicon member with respective edges of two or more housing hemispheres. This allows the housing to be quickly separated into multiple sections. Further, the housing may be held together by a temporary adhesive seal that is readily dissolved.

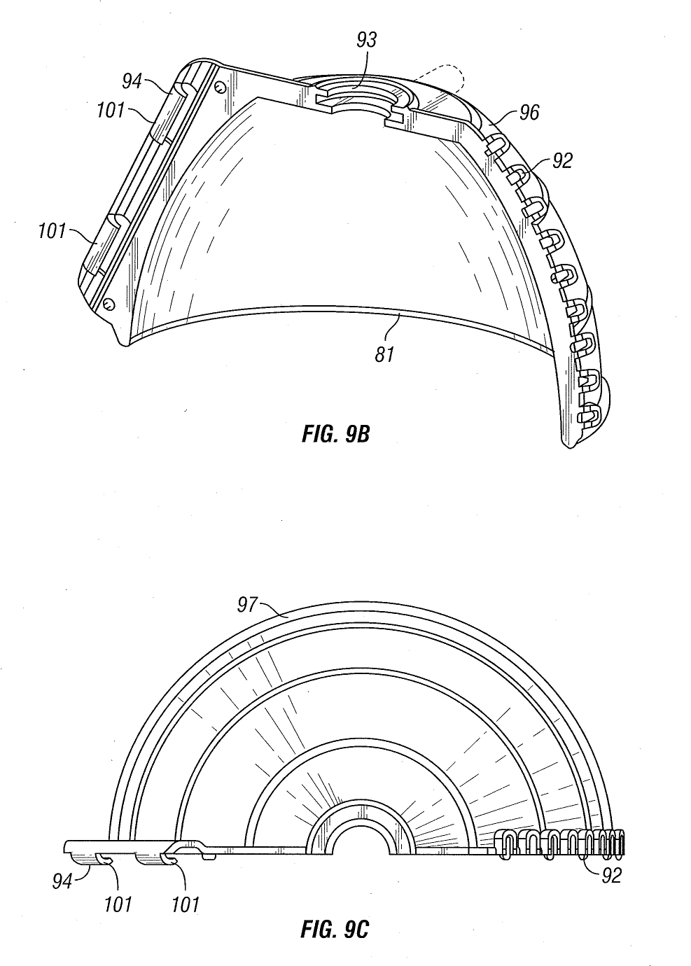

The housing used to connect to the invention is typically made of a rigid plastic or metal material that does not always produce adequate skin seals on patients having less elastic or thinner skin, e.g. the elderly or hirsute. FIG. 7 provides a side elevation view of a seal according to a further embodiment of the invention. In this embodiment of the invention, which operates in connection with a housing that is commonly a rigid clear plastic and that is not elastic or conformable, a rubber or elastomeric seal, gasket, or O-ring 81 is placed over a lip at the bottom edge of the housing 80 and makes contact with the patient's skin. The material for the seal may be bonded or press fit over the lip of the housing. Alternatively, a similar result may be obtained by applying a gel-like material on the edge of the housing that contacts the patient's skin or by placing such material directly on the skin of the patient. This embodiment of the invention not only improves the seal quality, but also prevents damage to the skin of the patients, especially those with thinner or less elastic skin.

For the purpose of this disclosure, the terms “zipper seal” and “zipper seal membrane” shall refer to a sealing apparatus for the mating hinge sections/mating members of the split shell vacuum housing as defined herein. In some embodiments of the invention, the zipper seal is an at-least partially elastic membrane that seals the mating membranes of a split shell vacuum housing. In some embodiments of the present invention, the zipper seal is stretchably coupled over a pair of mating membranes using an operating handle. Preferably, the zipper seal of the present invention provides an at least partially hermetic seal over the mating members.

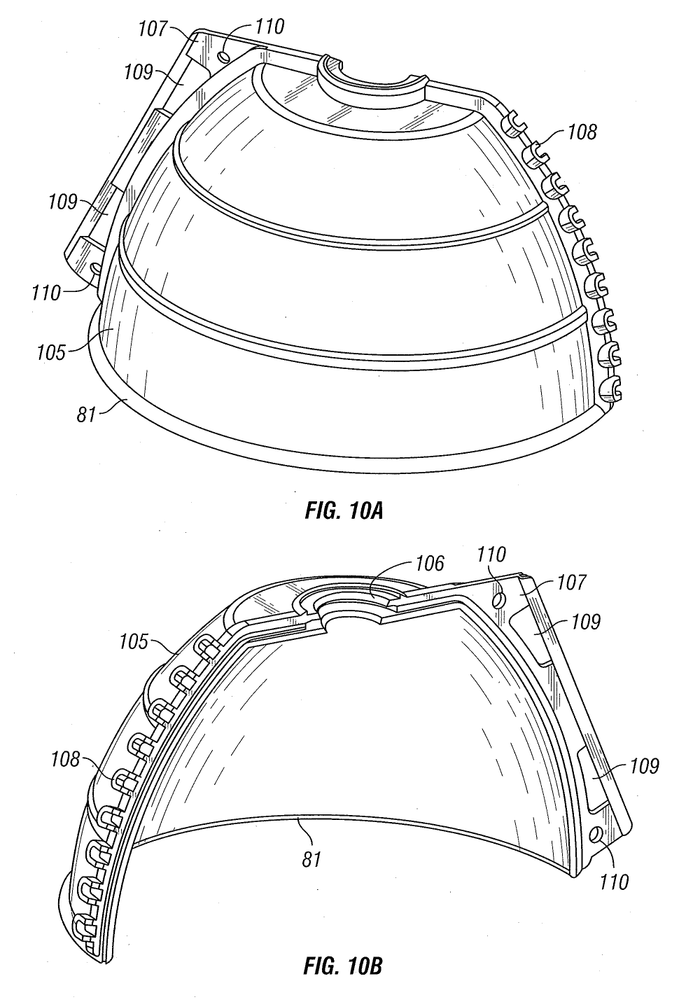

FIG. 8 is a perspective view of a split shell housing according to an embodiment of the invention. In FIG. 8, a housing 70 includes a first housing half 96 and a second housing half 97 which pivot together and apart via a hinge arrangement 72 comprised of mating hinged sections 94, 95. The housing of FIG. 8 includes a port 73, as discussed above in connection with the other embodiments of the invention. The port includes an annular recess 93 for receiving a port seal (discussed below). A base seal 81 functions as discussed above in connection with FIG. 7. The housing halves are secured together by means of a zipper seal 90 that includes an operating handle 91—the zipper seal engaged with mating protrusions on each of the housing halves to form a closure portion of a side seal assembly 71.

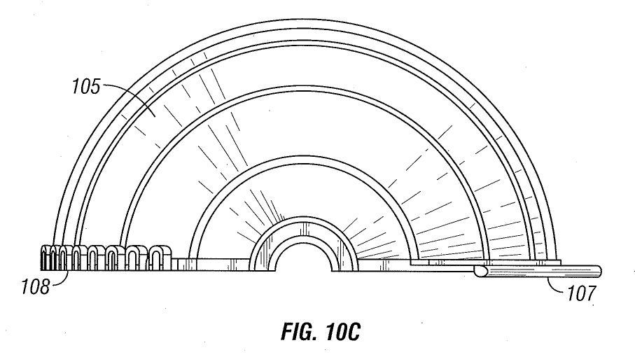

FIG. 9A shows a half of the housing 95 and, in particular, shows both the openings 100 and the hinge assembly 95 that receive mating portions 101 (FIG. 9B) from a hinge assembly portion 94 associated with a housing half 96. A plurality of slotted seal elements 92 are provided on housing portion 97 and are adapted to meet with complementary slots 99 formed on housing portion 96.

FIG. 9C provides a top plan view of the housing portion 96, showing the slotted seAL elements 92 and the hinge elements 101 of hinge portion 94.

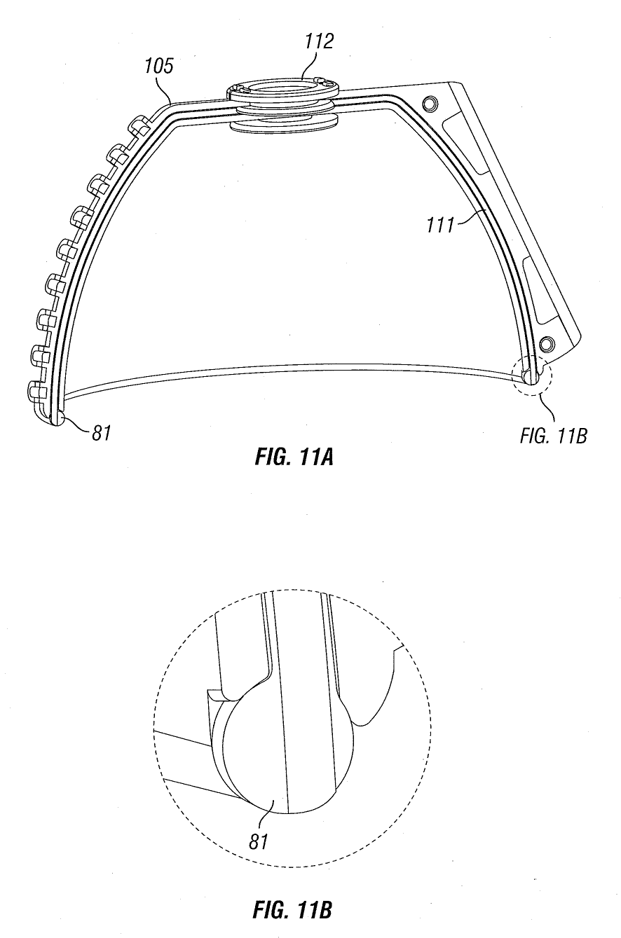

FIG. 10A shows a housing section 105 according to an alternate embodiment of the invention, in which a hinge assembly 107 includes openings 109 for receiving corresponding hinge elements (not shown), and which also shows a pair of locating apertures 110 for receiving complementary locating studs (not shown). An annular seating structure 106 is shown for seating a port seal. Additionally, a series of slotted members 108 are shown which receive complimentary slots from a second housing half for securing a housing. This embodiment of the invention functions in a way similar to that of FIG. 8. Thus, the closure mechanism 71 includes a zipper member 90 as shown in FIG. 8. The zipper member 90 is preferably made of a resilient material such that it conforms to the shape of the dome and flexibly expands and contracts to exert a clamping force to pull the two dome halves together and thus, hold the respective slots and pins of the two dome halves in engagement. The respecting slots and pins are preferably profiled with an inward taper to provide a gripping surface to the zipper seal. The zipper seal may be made of any suitable plastic material, such as Kapton® and Nylon®, as may be chosen by those skilled in the art.

FIG. 10B likewise shows the alternative embodiment of the invention. FIG. 10C is a further depiction of this embodiment showing a top plan view thereof.

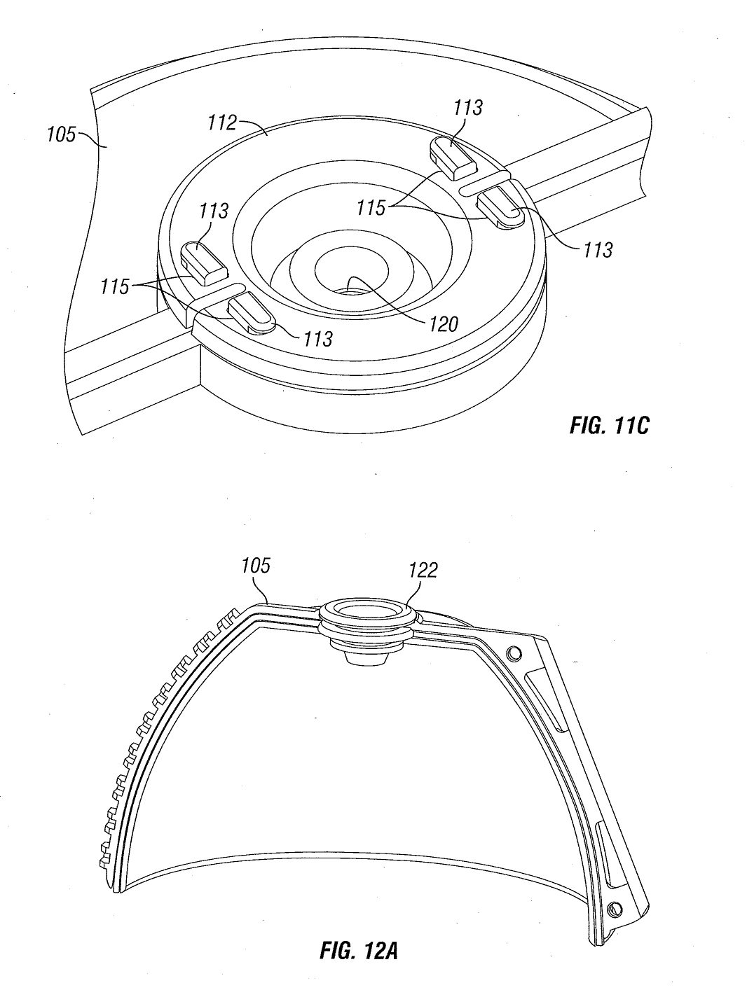

FIG. 11A shows a dome portion 105 including a port seal 112, an edge seal 111, and a base seal 81. The base seal 81 is shown in greater detail in perspective view of FIG. 11B. The seals should be sufficiently resilient to conform to the dome half edges, and compresses when the dome halves are brought together, such that an air-tight seal is achieved. Those skilled in the art will appreciate that any of such materials as rubber and synthetic materials may be chosen for this purpose.

FIG. 11C shows a perspective view of the port seal 112 engaged with the dome. In particular, the port seal 112 includes a plurality of apertures 115 that are adapted to engage with and surround projections 113. The projections shown are each associated with one half of the dome, such that there are four projections, with two projections on either side of the port on each section of the dome. This arrangement provides additional sealing of the dome halves and serves to hold them together, along with the zipper seal.

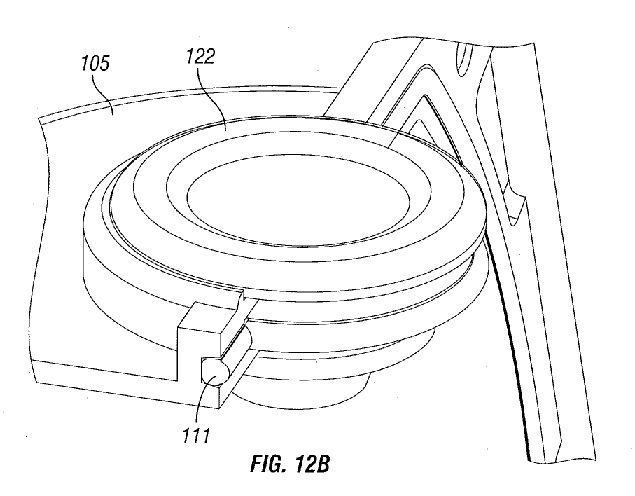

FIG. 12A is a side view of an alternative embodiment of the port seal 122. FIG. 12B shows a perspective view of the port seal 122 of FIG. 12A. A section view shows a detail of the edge seal 111.

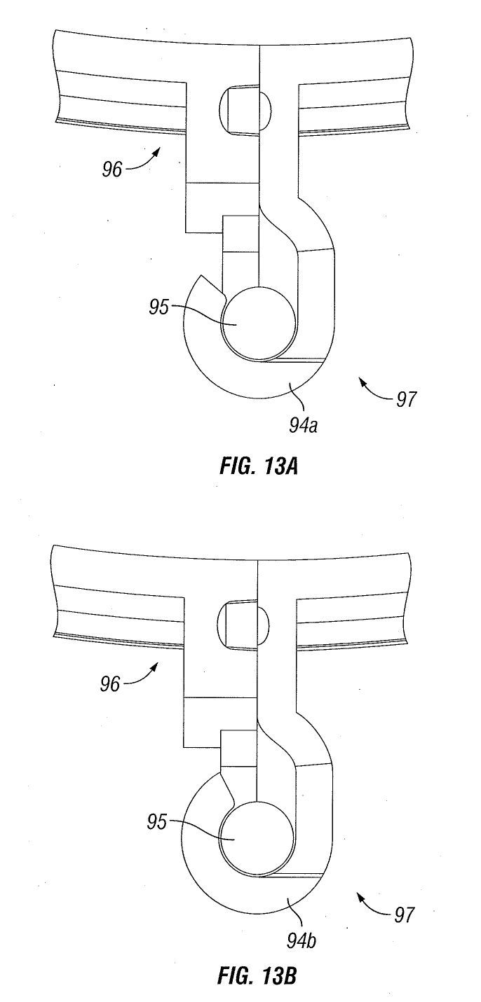

FIG. 13A is a top schematic view of the hinge assembly for dome portions 96 and 97 showing a slotted portion of the hinge member 95 and showing alternative versions of the hinge flange. In FIG. 13A, the hinge flange 94A provides 40 degrees of opening and closing of the dome portions, while FIG. 13B shows a flange portion 94B that allows 60 degrees of opening. Those skilled in the art will appreciate that various other arrangements are possible and are a matter of choice.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A medical device, comprising:
a split shell vacuum housing comprising a plurality of housing sections, at least one hinge, and at least one seal comprised of a complementary, abutting edge of each of said housing sections comprising mating members, and a zipper seal member that engages with said mating members to secure them in mating engagement;
means for introduction of a penetrator into said housing, comprising a port; and
a vacuum system comprising a vacuum source securely and sealably attached through the housing for advancing a patient's tissue onto the penetrator;
wherein said housing sections may be spread apart and/or separated along said hinge for removal of said housing from at least said penetrator.

2. A medical device, comprising:
a split shell vacuum housing comprising a plurality of housing sections and a bead and mating groove, interposed member, or adhesive bond that allows the sections of the housing to seal together and to separate apart;
said housing sections comprising at least one hinge, and at least one seal comprised of a complementary, abutting edge of each of said housing sections comprising mating members, and a zipper seal member that engages with said mating members to secure them in mating engagement;
a penetrator,
means for introduction of said penetrator into said housing, comprising valved port having a valve control and a port; and
a vacuum system comprising a vacuum source securely and sealably attached through the housing for advancing a patient's tissue onto the penetrator.

3. The medical device of claim 2, wherein the housing comprises a conforming housing seal along a perimeter thereof.

4. The medical device of claim 2, wherein the housing is manufactured from a strong non-collapsible material.

5. The medical device of claim 4, wherein the material is selected from a group consisting of plastic, plastic composite, rubber, rubber composite, fiberglass, epoxy, glass, and glass composite.

6. The medical device of claim 5, wherein the housing the material is translucent or transparent.

7. The medical device of claim 2, wherein the housing is separable into at least two sections.

8. The medical device of claim 2, wherein the housing is separable into two half sections, each section having a sealing edge to achieve a vacuum seal and a pass-through to receive the penetrator.

9. The medical device of claim 2, wherein the housing comprises a bell-shaped body having an opening, and a conforming port seal having a pass-through to receive the penetrator.

10. The medical device of claim 9, said port seal defining at least two slotted, transverse openings that are each adapted to receive a complementary member that projects from respective housing sections.

11. The medical device of claim 2, said mating members comprising a plurality of slotted members, each adapted to receive and retain within said slot a corresponding, complementary protruding member.

12. The medical device of claim 2, said mating members having a tapered profile that is adapted to engage with said zipper seal member.
